(19) United States
(12) Patent Application Publication
(10) Pub. No.: US 2016/0157827 A1
(43) Pub. Date: Jun. 9, 2016

(54) METHODS AND SYSTEMS FOR ULTRASOUND IMAGING

(71) Applicant: General Electric Company, Schenectady, NY (US)

(72) Inventor: Kjell Kristoffersen, Oslo (NO)

(21) Appl. No.: 14/559,804

(22) Filed: Dec. 3, 2014

(51) Int. Cl. A61B 8/08; A61B 8/00; A61B 8/14

(52) U.S. Cl. CPC A61B 8/5207; A61B 8/145; A61B 8/461; A61B 8/56; A61B 8/4494; A61B 8/54

(57) ABSTRACT

Systems and methods for automatically adjusting an analog time gain compensation utilized in ultrasound imaging systems are provided. In one embodiment, a method for ultrasound imaging comprises applying an analog gain to a first echo signal based on a depth and a direction of the first echo signal, wherein the analog gain is automatically adjusted based on a peak amplitude of a second echo signal in a preceding ultrasound image. In this way, a signal-to-noise ratio of echo signals may be optimized, thereby improving the quality of ultrasound images generated from the echo signals.

### FIELD

Embodiments of the subject matter disclosed herein relate to ultrasound imaging techniques, and more particularly, to adaptively controlling an analog time gain compensation.

### BACKGROUND

Modern ultrasound imaging systems employ digital beamforming based on digitized echo signals from an array of transducers to generate two- or three-dimensional B-mode images of tissue in which the brightness of a pixel or voxel is based on the intensity of the echo signals. To that end, such systems include analog-digital (A/D) converters to convert analog echo signals to digital echo signals for digital beamforming. However, the dynamic range of A/D converters may be much lower than that of the analog echo signals, so the A/D converters may be preceded by an analog stage with time-varying gain. This gain correction process is often referred to as analog time gain compensation (ATGC). Backscattered ultrasound signals, or echo signals, attenuate with depth, so ATGC in modern ultrasound imaging systems may comprise applying an analog gain that increases linearly in dB with depth, or time.

However, excessive analog gain may lead to saturation of the A/D converters. In some modes of operation, saturation may adversely affect the final ultrasound image. For example, signal clipping may cause significant 3^{rd} harmonic distortion, as well as 5^{th}, 7^{th}, and so on, which may cause blooming of strong echoes in 2^{nd} harmonic B-mode imaging. Conversely, analog gain that is too low may lead to loss of signal sensitivity and excessive noise.

### BRIEF DESCRIPTION

In one embodiment, a method for ultrasound imaging comprises applying an analog gain to a first echo signal based on a depth and a direction of the first echo signal, wherein the analog gain is automatically adjusted based on a peak amplitude of a second echo signal in a preceding ultrasound frame. In this way, a signal-to-noise ratio of echo signals may be optimized without saturating A/D converters, thereby improving the quality of ultrasound images generated from the echo signals.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE FIGURES

The present invention will be better understood from reading the following description of non-limiting embodiments, with reference to the attached drawings, wherein below:

FIG. 1 shows an ultrasound imaging system according to an embodiment of the invention.

FIG. 2 shows a high-level block diagram illustrating an ultrasound imaging system according to an embodiment of the invention.

FIG. 3 shows a high-level block diagram illustrating an analog time gain compensation controller according to an embodiment of the invention.

FIG. 4 shows an example graphical model of control points for updating an analog time gain compensation profile according to an embodiment of the invention.

FIG. 5 shows a high-level flow chart illustrating an example method for adjusting an analog time gain compensation profile for a given ultrasound frame according to an embodiment of the invention.

FIG. 6 shows a graph illustrating example analog time gain compensation limits according to an embodiment of the invention.

FIG. 7 shows a high-level flow chart illustrating an example method for actively controlling an analog time gain compensation during an ultrasound scanning session according to an embodiment of the invention.

### DETAILED DESCRIPTION

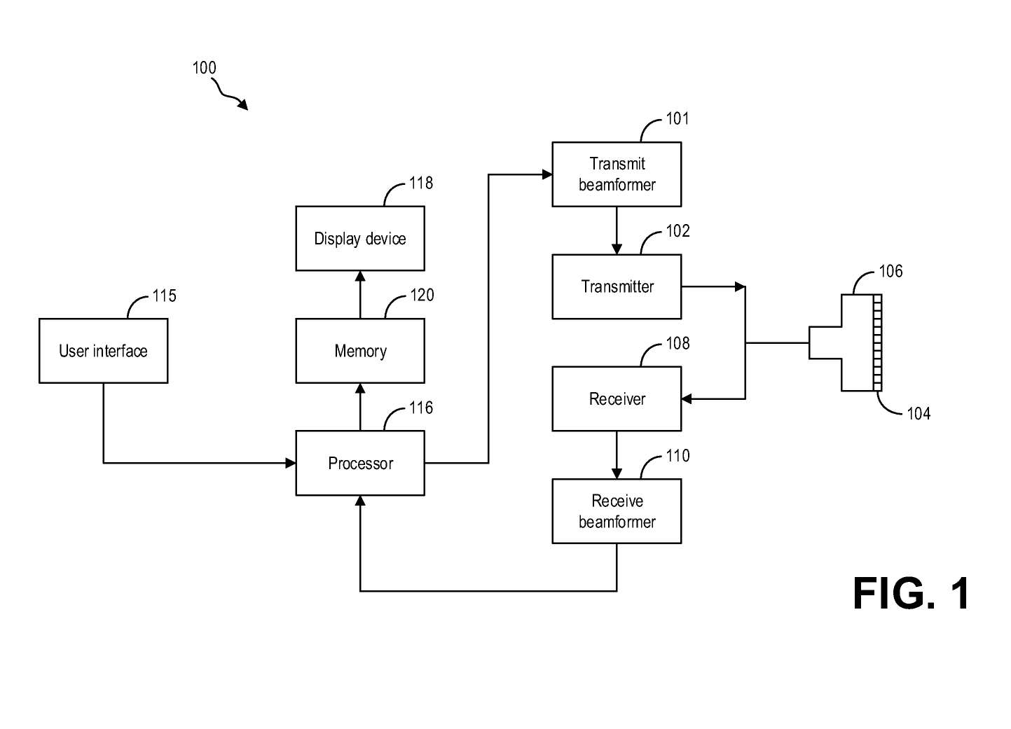

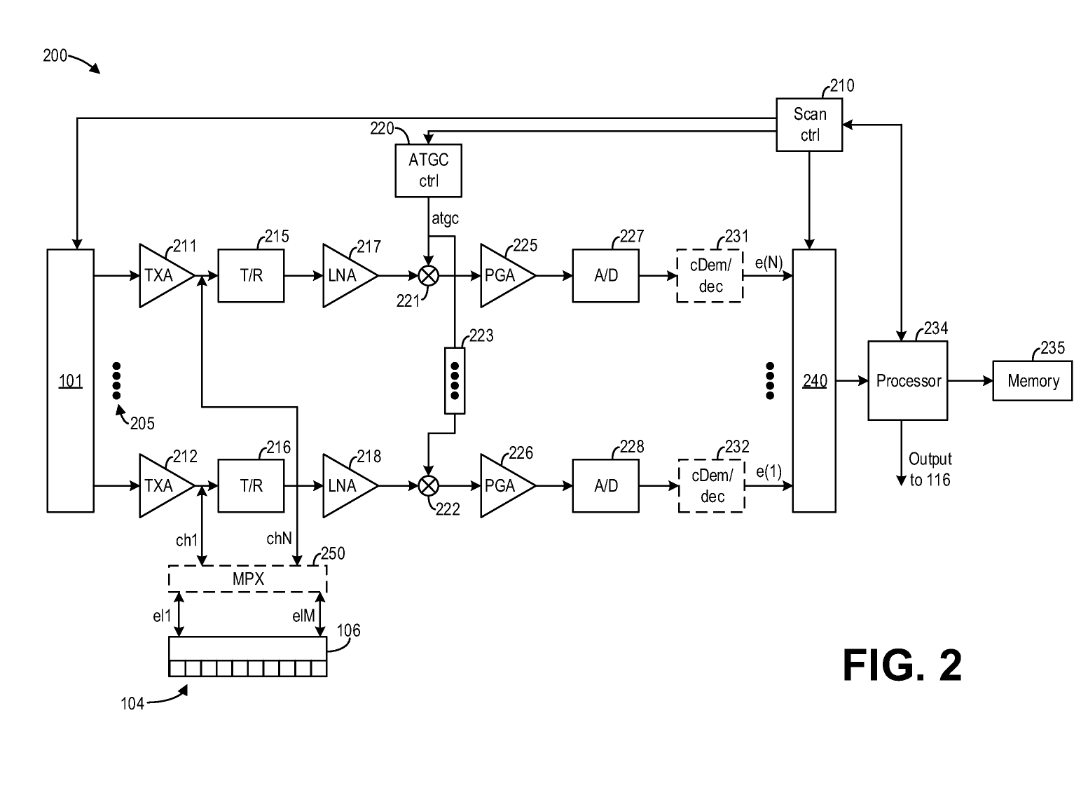

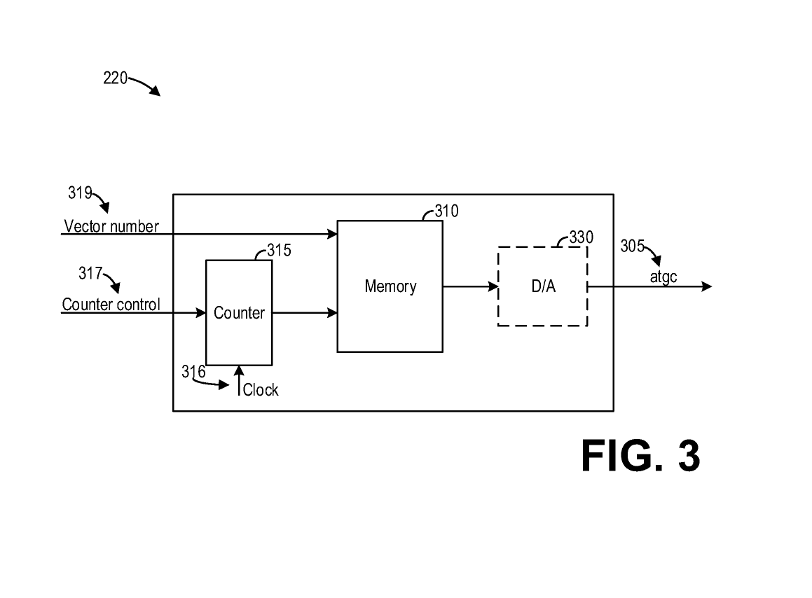

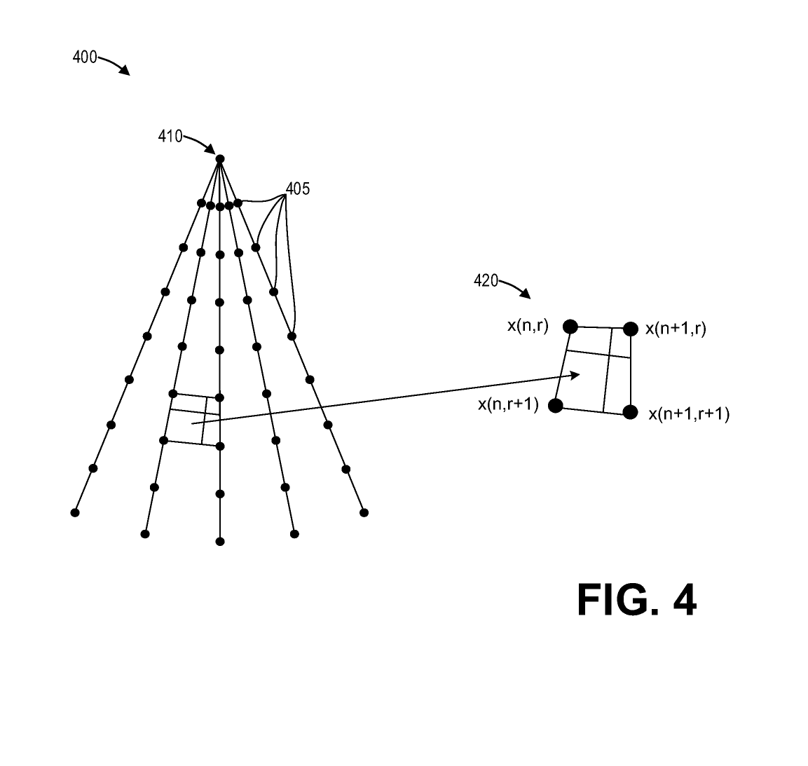

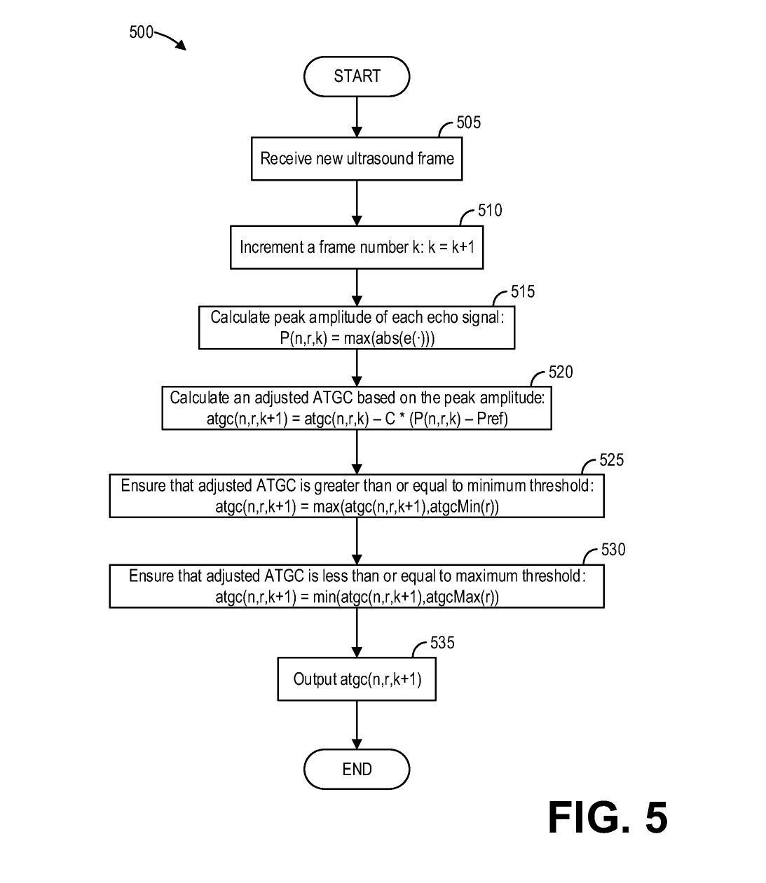

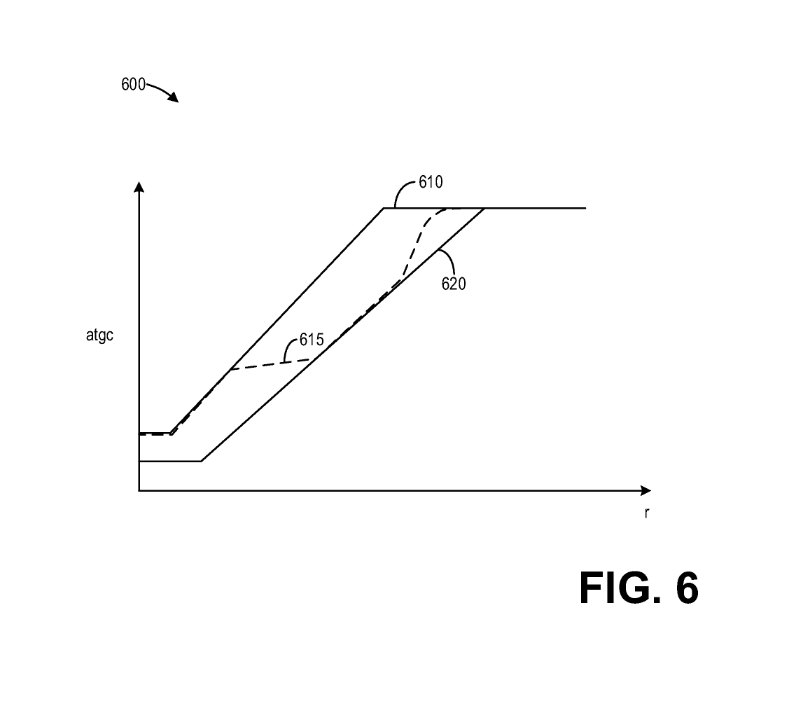

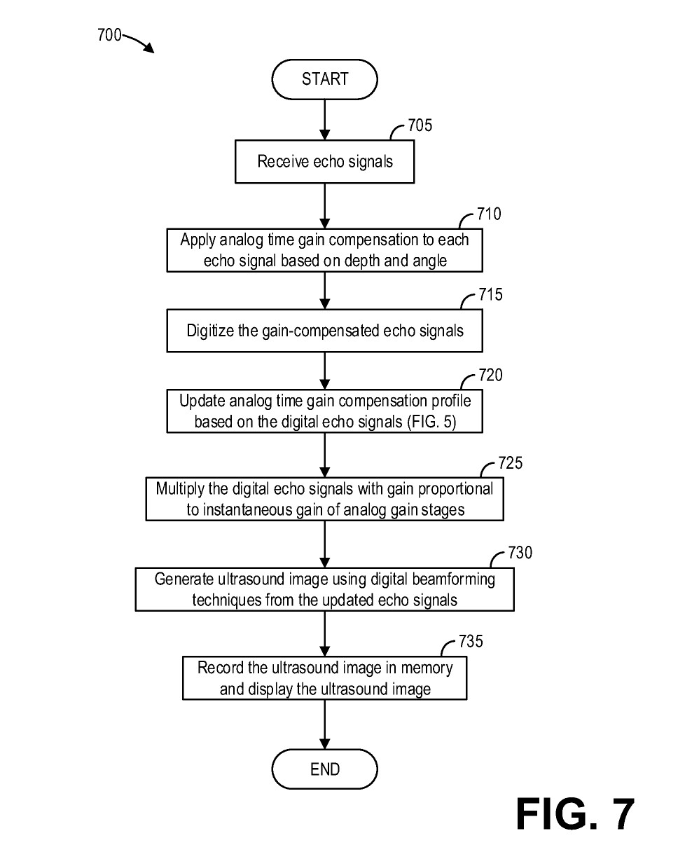

The following description relates to various embodiments of ultrasound imaging techniques. In particular, methods and systems for automatically adjusting an analog time gain compensation (ATGC) profile are provided that improve control of the ATGC in order to improve signal-to-noise ratio (SNR) of echo signals while balancing other issues. An ultrasound imaging system such as the system shown in FIGS. 1 and 2 may include an ATGC controller, such as the controller shown in FIG. 3, configured to apply an analog gain to echo signals. The analog gain may compensate for attenuation of the echo signals caused by tissue and strong scatterers, as well as diffraction effects. The peak amplitudes of gain-compensated echo signals originating from control points, such as those depicted in FIG. 4, may be used to adjust the analog gain for subsequent ultrasound frames using the method shown in FIG. 5. Adjustments to the analog gain may be limited by a maximum and minimum threshold, such as the thresholds depicted in FIG. 6, in order to prevent saturation of A/D converters and maintain a baseline SNR. A method for generating ultrasound images with dynamically-adjusted gain compensation is shown in FIG. 7.

FIG. 1 is a schematic diagram of an ultrasound imaging system 100 in accordance with an embodiment of the invention. The ultrasound imaging system 100 includes a transmit beamformer 101 and a transmitter 102 that drive elements 104 of a transducer array, possibly located inside a probe, 106 to emit pulsed ultrasonic signals into a body (not shown). According to an embodiment, the transducer array 104 may be a one-dimensional array. However, in some embodiments, the transducer array 104 may be a two-dimensional matrix array. Still referring to FIG. 1, the pulsed ultrasonic signals are back-scattered from structures in the body, like blood cells or muscular tissue, to produce echoes that return to the elements of the array 104. The echoes are converted into electrical signals, or ultrasound data, by the elements of the array 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. A user interface 115 may be used to control operation of the ultrasound imaging system 100, including to control the input of patient data, to change a scanning or display parameter, and the like. The user interface 115 may include one or more of the following: a rotary, a mouse, a keyboard, a trackball, hard keys linked to specific actions, soft keys that may be configured to control different functions, and a graphical user interface displayed on the display device 118.

The ultrasound imaging system 100 also includes a processor 116 to control the transmit beamformer 101, the transmitter 102, the receiver 108, and the receive beamformer 110. The processer 116 may be a digital processor coupled with memory and may be in electronic communication with the probe 106. For purposes of this disclosure, the term “electronic communication” may be defined to include both wired and wireless communications. The processor 116 may control the probe 106 to acquire data. The processor 116 controls which of the elements 104 are active and the shape of a beam emitted from the probe 106. The processor 116 is also in electronic communication with a display device 118, and the processor 116 may process the data into images for display on the display device 118. The processor 116 may include a central processor (CPU) according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment, the demodulation can be carried out earlier in the processing chain. The processor 116 is adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. For example, an embodiment may acquire images at a real-time rate of 7-20 volumes/sec. The ultrasound imaging system 100 may acquire 2D data of one or more planes at a significantly faster rate. However, it should be understood that the real-time volume-rate may be dependent on the length of time that it takes to acquire each volume of data for display. Accordingly, when acquiring a relatively large volume of data, the real-time volume-rate may be slower. Thus, some embodiments may have real-time volume-rates that are considerably faster than 20 volumes/sec while other embodiments may have real-time volume-rates slower than 7 volumes/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a volume-rate of, for example, 10 Hz to 30 Hz. Images generated from the data may be refreshed at a similar frame-rate. Other embodiments may acquire and display data at different rates. For example, some embodiments may acquire data at a volume-rate of less than 10 Hz or greater than 30 Hz depending on the size of the volume and the intended application. A memory 120 is included for storing processed volumes of acquired data. In an exemplary embodiment, the memory 120 is of sufficient capacity to store at least several seconds worth of volumes of ultrasound data. The volumes of data are stored in a manner to facilitate retrieval thereof according to its order or time of acquisition. The memory 120 may comprise any known data storage medium. For the purposes of this disclosure, an ultrasound image may refer to an ultrasound frame for two dimensions or an ultrasound volume (comprising a set of frames) for three dimensions.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. The image lines and/or volumes are stored and timing information indicating a time at which the data was acquired in memory may be recorded. The modules may include, for example, a scan conversion module to perform scan conversion operations to convert the image volumes from beam space coordinates to display space coordinates. A video processor module may be provided that reads the image volumes from a memory and displays an image in real time while a procedure is being carried out on a patient. A video processor module may store the images in an image memory, from which the images are read and displayed.

In one embodiment, the processor 116 may be configured to adaptively adjust an analog time gain compensation (ATGC) profile applied to an echo signal to maximize a SNR of the echo signal without saturating an A/D converter. An ultrasound imaging system configured to adjust an ATGC profile is described further herein and with regard to FIG. 2.

FIG. 2 shows a high-level block diagram illustrating the acquisition part of an example ultrasound imaging system 200 in accordance with the present disclosure. In particular, ultrasound imaging system 200 may adjust an analog gain applied to echo signals received during an ultrasound scanning session. Ultrasound imaging system 200 may include N identical analog channels, however for simplicity only channel 1 (ch1) and channel N (chN) are explicitly depicted while the additional identical channels 2 through N-1 are referenced by 205.

Processor 234 may command an ultrasound scan via scan controller 210. In one embodiment this may be a standalone computer, such as a Graphical Processor Unit (GPU) communicating with the processor 116. In another embodiment this could be the same computer as the processor 116. Scan controller 210 may in turn command transmit beamformer 101 to prepare one or more ultrasound beams based on operator input received via user interface 115. Transmit beamformer 101 may determine a delay pattern and pulse train that sets a desired transmit beam focal point. The outputs of the transmit beamformer 101 may be amplified by a transmit amplifier (TXA) 212. TXA 212 may comprise a high-voltage transmit amplifier that drives the transducer elements 104 of the probe 106. Transmit beams in each channel, such as ch1 and chN, may be directed to the transducer 106 by transmit/receive (T/R) switches 215 and 216. T/R switches 215 and 216 may comprise, for example, a diode bridge that blocks the high-voltage transmit pulses from damaging the receiver components. Multiplexer (MPX) 250 may optionally be included in ultrasound imaging system 200 to direct transmit signals to different transducer elements 104 and/or the echo signals from the different transducer elements 104 to the appropriate channel. Transmit and receive signals communicated between MPX 250 and transducer 106 are shown by e11 and e1M, where M may be significantly larger number than N. Examples are M=192 and N=128, however in some examples M and N may comprise different numbers than 192 and 128, respectively.

After ultrasonic transmit beams are emitted into the subject and corresponding echoes are received by transducer 106, the echo signals produced by transducer elements 104 pass through the T/R switches 215 and 216 to enter an amplification stage. In particular, echo signals may pass through low-noise amplifiers (LNA) 217 and 218 and programmable gain amplifiers (PGA) 225 and 226 which apply a constant gain. As a non-limiting example, LNAs 217 through 218 and PGAs 225 through 226 may implement apodization functions, or spatial windowing to reduce sidelobes in the beam (not shown in FIG. 2). In another example this function may be performed in the digital domain.

Furthermore, an analog time gain may be applied to the echo signals. The ultrasound waves are attenuated in proportion to the distance that the sound waves travel to reach a reflector, plus the distance that the resulting echoes travel back to reach the transducer 106. Thus, the deeper the penetration of the ultrasound waves, the greater the attenuation. Consequently, the strength of received echoes becomes weaker with increased depth and time of travel. In order to compensate for the decreased strength of echo signals caused by attenuation and beam diffraction, ATGC controller 220 may supply a gain signal atgc to each channel to compensate for attenuation. Specifically, the gain signal atgc may increase as echoes are received from deeper tissues or equivalently with time. Signal atgc may be multiplied by each individual channel, for example, at multiplicative junctions 221, 222, and 223. The multipliers' response to the control signal atgc may have an exponential characteristic, i.e. the gain of the channel signals increases exponentially with a linear increase of the control atgc. In this way, the dynamic range over which the echoes may be heard may be increased.

In addition to attenuation due to travel time through the tissue, the amplitude of the ultrasound data coming from the array elements will vary depending on the presence of scatterers in the body. Strong scatterers will give a stronger echo relative to weak scatterers at the same depth. ATGC controller 220 may adjust the gain signal atgc to account for such variations in signal amplitude. In particular, the gain signal labeled atgc in FIG. 2 may be automatically decreased for directions and depths containing strong scatterers, and increased for directions and depths without strong scatterers. In this way, signal-to-noise ratio for echo signals containing weak scatterers may be increased, without associated clipping of signals from strong scatterers, thereby improving the quality of the final ultrasound image. A method for automatically adjusting an ATGC profile to account for the presence or absence of strong scatterers is described further herein and with regard to FIG. 5.

After the gain amplification stage, the amplified analog echo signals may be converted into digital echo signals via A/D converters 227 and 228. Complex demodulator/decimator (cDem/dec) 231 and 232 may be optionally included in ultrasound imaging system 200 for data reduction and extracting phase and amplitude information from the digitized channel data.

After conversion and optional demodulation/decimation, data packing and communication module 240 may prepare the digital channels e(1) through e(N) for digital receive beamforming by processor 234. Processor 234 may comprise, for example, a GPU or a CPU configured to perform digital (e.g., software) receive beamforming, and delivers its beamformed output data to the processor 116.

In one embodiment, the processor 234 monitors the digitized outputs e(1) through e(N). For each point in space, the processor 234 may maximize the gain signal atgc while avoiding saturation of the A/D converters 227 and 228. This is accomplished via a feedback loop between processor 234 and ATGC controller 220, which may be updated for each new ultrasound frame as described further herein with regard to FIG. 5.

The channel output from individual A/D converters may be individually monitored and/or processed by processor 234. In one embodiment, processor 234 may multiply channel data such as e(1) and e(N) by a number inversely proportional to the instantaneous gain of the analog gain stages for each channel, where the gain stage for channel 1, for example, may include the LNA 218, atgc at 222, and the PGA 226. In this way, no further downstream gain compensations may be applied when the ATGC matrix changes dynamically in time or across the imaged field or volume.

FIG. 3 shows a high-level block diagram illustrating an example analog time gain compensation (ATGC) controller 220 in accordance with the present invention. As shown, ATGC controller 220 may comprise a memory 310 and a counter 315. In embodiments where the output atgc 305 from ATGC controller 220 may comprise an analog signal, ATGC controller 220 may further include a digital-analog (D/A) converter 330 to convert the digital signal into an analog signal. ATGC controller 220 may be included in the systems depicted in FIGS. 1 and 2.

Memory 310 may store an ATGC matrix comprising an ATGC curve for every range and transmit vector. Memory 310 may comprise, for example, a RAM, however in some embodiments memory 310 may comprise any suitable data storage medium. Memory 310 may receive input from counter 315 as well as a vector number 319 from scan controller 210, and memory 310 may output a gain value based on the counter 315 output and the vector number 319. In this way, memory 310 may provide an analog time gain compensation for a scanning session, where the gain applied to a particular echo signal may depend on the depth and direction of the echo signal.

Counter 315 may provide a register of the time of flight of ultrasound waves during scanning, referred to herein as the range of an echo signal. To that end, counter 315 may be coupled to a clock 316 and may receive input from scan controller 310 in the form of a counter control signal 317.

In one embodiment, memory 310 may be updated by the processor 234 in real time during scanning. For example, the processor 234 may monitor the digital channel output and determine if the gain output by ATGC controller 220 may be increased or reduced based on the digital channel output. The processor 234 may then update the ATGC value in memory 310 such that the SNR of the subsequently processed signal is maximized.

In some examples, the processor 234 may process each point of the scanned region to update the memory 310. In other examples, processor 234 may process, or sample, a subset of scan points to obtain updated ATGC values for those scan points, and may use interpolation to obtain updated ATGC values for unprocessed scan points. FIG. 4 shows a graphical illustration of an example configuration 400 of control points 405 for adjusting an ATGC matrix stored in memory 310. The ATGC may be adaptively controlled for these control points 305, and a higher resolution ATGC profile may be generated through linear interpolation. The interpolation may be carried out by the processor 234. In an alternative embodiment, the interpolation may be carried out by a dedicated hardware structure that performs the interpolation in real time.

Each control point 405 may correspond to a pair of indices, such as a lateral transmit beam index n and a range index r. For example, the lateral index and range index of control point 410 may equal zero, where control point 410 may comprise a point in the tissue closest to the transducer 106. The indices may increase as illustrated by the subset 420 of control points. For example, the lateral index n may increase by one for each transmit beam direction, while the range index r may increase based on the distance of a control point from control point 410. As such, the lateral index n may correspond to specified transmit vector numbers, while the range index r may correspond to a depth or time of travel of an echo signal.

FIG. 5 shows a flow chart illustrating an example method 500 for updating an ATGC profile for a given frame in accordance with the current disclosure. Method 500 may be carried out by processor 234 in combination with one or more hardware components and may be stored as executable instructions in memory 235. In some embodiments the memory 235 may be the same as memory 120. The processor 234 may perform a peak detection of the maximum echo amplitude across participating channels and the spatial neighborhood (over transmit vectors and range samples) that belong to control points of interest, possibly in combination with hardware, such as the various hardware components described herein.

Method 500 may begin at 505. At 505, method 500 may include receiving a new ultrasound frame. The ultrasound frame may comprise, for example, a plurality of echo signals. Continuing at 510, method 500 may include incrementing the frame number k by one, or setting k−k+1.

At 515, method 500 may include determining the maximum peak amplitude of each echo signal based on the origin of each echo signal, for example based on the lateral index n and the range index r of each echo signal. As described above with regard to FIG. 4, in one embodiment method 500 may include calculating the peak amplitude for a subset of echo signals originating from a set of control points 405, thereby reducing the computational expense of step 515. The peak amplitude P(n,r,k) may be set to the maximum absolute value of the channel signal e(.), or P(n,r,k)=max(abs(e(.))), where the dot in e(.) corresponds to a particular channel.

At 520, method 500 may include calculating an adjusted ATGC for a next ultrasound frame, or atgc(n,r,k+1), based on the peak amplitude. In particular, the ATGC for the next ultrasound frame atgc(n,r,k+1) may be set to the ATGC for the current frame atgc(n,r,k) minus a difference between the peak amplitude P(n,r,k) and a reference peak value Pref, where the difference is scaled by a constant C. The constant C may be selected to control the speed of adaptation. In this way, if the amplitude P(n,r,k) exceeds the reference value Pref, the analog gain will be reduced for the next frame.

At 525, method 500 may include ensuring that the updated analog time gain compensation for the next ultrasound frame is greater than or equal to a minimum limit or threshold. For example, the updated analog time gain compensation calculated at 520, or atgc(n,r,k+1), may be compared to a minimum value atgcMin(r). A function max( ) may return the larger value of the two values. In this way, if the updated analog time gain compensation calculated at 520 is below a minimum threshold set by atgcMin(r), a minimum value may be selected instead of the value calculated at 520. Otherwise, the updated analog time gain compensation may remain equal to the value calculated at 520.

At 530, method 500 may include ensuring that the updated analog time gain compensation for the next frame is less than or equal to a maximum limit or threshold. For example, the updated analog time gain compensation calculated at 525 may be compared to a maximum value atgcMax(r). A function min( ) may return the smaller value of the two values. In this way, if the updated analog time gain compensation atgc(n,r,k+1) calculated at 525 is larger than a maximum threshold set by atgcMax(r), a maximum value may be selected instead of the value calculated at 525. Otherwise, the updated analog time gain compensation may remain equal to the value calculated at 525.

At 535, method 500 may include outputting the updated ATGC values calculated at 530 for each echo signal for the next frame. The updated ATGC values may be output, for example, to memory 310. In this way, the gain of the analog signal chain may be maximized under the constraint of avoiding signal saturation, so that the SNR of echo signals in subsequent ultrasound frames may be optimized. In some examples, method 500 may further include interpolating ATGC values for echo signals not originating from the control points 405, and such interpolated ATGC values may also be output at 535. Method 500 may then end.

As discussed herein above with regard to steps 525 and 530, functions atgcMin(r) and atgcMax(r) may set limits on the minimum and maximum gain provided to echo signals based on the distance given by the index r. In this way, excessive control is avoided by a preset maximum and minimum gain for each given range. FIG. 6 shows a graph 600 illustrating example maximum and minimum gain limits in accordance with the current disclosure. Graph 600 includes plots 610 and 620, where plot 610 corresponds to a maximum gain limit atgcMax(r) and plot 620 corresponds to a minimum gain limit atgcMin(r). As depicted, the preset gain limits may be a combination of linear segments. In some examples, however, dependent of the transfer function from control to gain, the gain limits may be exponential. Furthermore, as shown by plots 610 and 620, the gain limits may increase over a range of r values and may remain constant outside of that range. Plot 615 shows an example of what an actual gain profile may look like for a vector.

FIG. 7 shows a high-level flow chart illustrating an example method 700 for adaptively controlling an analog time gain compensation (ATGC) in accordance with the current disclosure. In particular, method 700 relates to adjusting an analog time gain compensation applied to echo signals based on a depth and direction of the echo signals to maximize a signal-to-noise ratio without saturating an analog-digital converter. The adjustment may occur from frame to frame during an ultrasound scan. Method 700 may be carried out by the systems and components depicted in FIGS. 1 through 3, however the method may be applied to other systems without departing from the scope of the current disclosure.

Method 700 may begin at 705. At 705, method 700 may include receiving a set of echo signals. At 710, method 700 may include applying an ATGC to each echo signal based on the origin of the echo signal, that is, where the ultrasonic transmit wave reflected within the subject, or the depth and angle of the echo signal. In some examples, the ATGC applied to a particular echo signal may be adjusted based on a peak amplitude of a previous echo signal from the same origin. At 715, method 700 may include digitizing the gain-compensated echo signals, for example using the A/D converters 227 and 228.

At 720, method 700 may include updating an ATGC profile based on the digital echo signals. As a non-limiting example, the ATGC profile may be updated as described herein above with regard to FIG. 5. For example, the maximum absolute value, or peak amplitude, of each echo signal may be used to determine an updated ATGC value that may be applied to a subsequent echo signal from the same origin place, where such an ATGC value may be stored, for example, in memory 310 of ATGC controller 220. The updated ATGC value may then be compared to maximum and minimum limits, such as those depicted by plots 610 and 620 in FIG. 6, where the maximum and minimum limits are specified based on the limitations of the A/D converters responsible for converting the analog echo signals into digital echo signals.

Thus, updating an ATGC profile based on the digital echo signals may comprise recording an adjusted ATGC value in memory 310 for subsequent application to succeeding echo signals. In this way, the SNR of subsequently received echo signals, and therefore the image quality of subsequently generated ultrasound images, may be automatically optimized

Continuing at 725, method 700 may include multiplying the digital echo signals with a gain proportional to the instantaneous gain of analog gain stages preceding the A/D converter. In this way, additional downstream compensations for adjusted gains may not be necessary. At 730, method 700 may include generating an ultrasound image using digital beamforming techniques from the gain-adjusted digital echo signals. At 735, method 700 may include recording the ultrasound image in memory, such as memory 120, and displaying the ultrasound image, for example using the display 118. Method 700 may then end.

As a non-limiting illustrative example, consider a single ultrasound scanning session, or scan in accordance with the current disclosure. In particular, in order to generate a single ultrasound frame, or image, during such a scan, a plurality of ultrasonic transmit waves may be emitted from transducer elements of a transducer probe into a patient. The plurality of ultrasonic transmit waves travel through the body of the patient, and eventually each of the ultrasonic transmit waves reflects at different locations of one or more structures within the patient. The reflected ultrasonic waves, or echoes, travel back to the transducer probe. As the echoes reach the transducer elements of the transducer probe, the transducer elements convert the ultrasonic echoes into analog electrical signals, or echo signals. A different analog gain may be applied to each echo signal to account for different amounts of attenuation due to the different amounts of distance (and therefore, time) traveled by each echo. Initially, the analog gain applied to each echo signal may comprise a feed-forward analog gain initially stored as an analog gain matrix in the memory of an analog time gain compensation controller configured to apply the analog gain to the echo signals. This gain profile could, for example, be the minimum limit 620. The gain-compensated echo signals may then be digitized by A/D converters and the digital echo signals may be sent to a processor. The processor may evaluate each of the digital echo signals to determine if the analog gain may be increased or reduced. In one example, the processor may process a subset of the digital echo signals, where each digital echo signal in the subset reflected at a pre-specified control point within the patient, to compute an adjusted analog gain for each echo signal in the subset based on the signal strength of each echo signal and the limitations of the A/D converters. The processor may then interpolate adjusted analog gains for the complement of the subset. The processor may then update the analog gain matrix with the adjusted analog gains, including the directly computed adjusted analog gains and the interpolated analog gains. In some examples, the processor may apply small gain adjustments to the digital echo signals based on the instantaneous analog gain, thereby taking into account, to some extent, any substantial adjustments to the analog gain matrix. The processor may then use digital beamforming techniques to generate and output to memory and/or a display a single ultrasound frame from the digital echo signals. Meanwhile, the transducer probe may emit a second plurality of ultrasonic transmit waves in order to form a second ultrasound frame as just described. A second set of echo signals produced by this second plurality of ultrasonic transmit waves may then undergo analog time gain compensation using the adjusted analog gains of the updated analog gain matrix. The second set of gain-compensated echo signals may feature an improved signal-to-noise ratio compared to the first set of gain-compensated echo signals due to the adaptive control of the analog time gain compensation. After digital conversion and digital beamforming, the processor may generate and output a second ultrasound frame. This second ultrasound frame may feature an improved image quality with a reduced number of artifacts compared to the first ultrasound frame due to the improved signal-to-noise ratio of the digital echo signals. Furthermore, the processor may evaluate the second set of digital echo signals to determine additional adjustments to the analog gain matrix as described above. As a result, a third ultrasound frame may feature an improved image quality with a reduced number of artifacts compared to the second ultrasound frame, and/or compensating for new changing positions of the scatterers within the image frame caused by probe motion and/or motion of the target itself, such as in the case of a beating heart. This process may repeat throughout the ultrasound scan. In this way, the signal-to-noise ratio of echo signals may kept at an optimal level throughout an ultrasound scan. As a result, the image quality of each ultrasound frame may improve. Furthermore, the system continuously adapts to any changes during the scan.

The technical effect of the disclosure may include an automatic adjustment of analog time gain compensation applied to ultrasound echo signals based on the signal strength of preceding ultrasound echo signals. Another technical effect of the disclosure may include an improved signal-to-noise ratio of echo signals. Yet another technical effect of the disclosure may include an increased dynamic range of the digitized echo signal strength. Another technical effect of the disclosure may include the generation of ultrasound images with improved image quality.

In one embodiment, a method for ultrasound imaging comprises applying an analog gain to a first echo signal based on a depth and a direction of the first echo signal, wherein the analog gain is automatically adjusted based on a peak amplitude of a second echo signal in a preceding ultrasound image. In one example, the second echo signal originates from the same depth and direction and covers a same spatial neighborhood as the first echo signal. The method further comprises generating an ultrasound image based on the first echo signal and displaying the ultrasound image on a display.

In one example, adjusting the analog gain based on the peak amplitude comprises calculating a difference between the peak amplitude and a reference amplitude, and subtracting a value proportional to the difference from an analog gain applied to the second echo signal. In another example, the analog gain is further adjusted based on limits of an analog-digital converter configured to digitize the first echo signal. For example, adjusting the analog gain based on the limits comprises setting the analog gain to a maximum limit if the analog gain is above the maximum limit, and setting the analog gain to a minimum limit if the analog gain is below the minimum limit.

The method further comprises multiplying the second echo signal by a value proportional to an instantaneous gain applied to the first echo signal.

In another embodiment, a method for ultrasound imaging comprises applying a first analog gain to a first echo signal based on the depth and direction of the first echo signal, measuring a peak amplitude of the first echo signal, adjusting a second analog gain applied to a second echo signal based on the peak amplitude, generating a first ultrasound image based on the first echo signal and a second ultrasound image based on the second echo signal, and displaying the first ultrasound image and the second ultrasound image in succession.

In one example, measuring the peak amplitude is performed responsive to the first echo signal originating from a specified control point. The method further comprises interpolating a third analog gain applied to a third echo signal based on the adjusted second analog gain.

In one example, the first echo signal is converted to a first digital echo signal after applying the first analog gain and prior to measuring the peak amplitude. In another example, the second echo signal is converted to a second digital echo signal after applying the second analog gain and prior to generating the second ultrasound image.

In yet another example, generating the first and second ultrasound images comprises applying digital beamforming techniques respectively to the first echo signal and the second echo signal. In another example, displaying the ultrasound images comprises transmitting the ultrasound images to a display device.

The method further comprises multiplying the first echo signal by a value proportional to an instantaneous gain applied to the second echo signal prior to generating the first ultrasound image.

In yet another embodiment, an ultrasound imaging system comprises: a transducer array including a plurality of array elements, the transducer array adapted to transmit a plurality of ultrasound waves and receive a plurality of echoes; a display device configured to display an ultrasound image; a gain controller comprising a memory, the memory configured with an analog gain matrix, the gain controller configured to apply an analog gain output by the memory to each of the plurality of echoes; and a processor configured with computer-readable instructions in non-transitory memory that when executed cause the processor to update the analog gain matrix based on a peak amplitude of each of the plurality of echoes and generate the ultrasound image based on the plurality of echoes.

In one example, the analog gain matrix comprises a table of analog time gain compensation values, wherein each of the analog time gain compensation values corresponds to a particular range and vector number.

In another example, the gain controller further comprises a counter configured to provide a range of each of the plurality of echoes to the memory, and the analog gain applied to each of the plurality of echoes is based on the range of each of the plurality of echoes. In yet another example, the gain controller further comprises a digital-analog converter configured to convert a digital gain value from the memory into the analog gain.

In one example, the processor is further configured with computer-readable instructions in the non-transitory memory that when executed cause the processor to compute a first adjusted analog gain based on a specified echo and interpolate a second adjusted analog gain based on the first adjusted analog gain. In such an example, updating the analog gain matrix comprises recording the first adjusted analog gain and the second adjusted analog gain to the memory.

Other modifications may be added to enhance the functionality of the adaptive analog atgc control. For example, it may be advantageous to low-pass filter the atgc gain matrix in 2D (radial/lateral) space, to avoid discontinuities in the noise background of the image. In this case the gain of a spatial point will depend not only on the amplitude of the echoes from its own history, but also on the echo history of its spatial neighborhood. It is also straightforward for someone skilled in the art to extend the method to volumetric acquisition of ultrasound data. This can be done by adding an extra spatial dimension to the ATGC control.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for ultrasound imaging, comprising:
applying an analog gain to a first echo signal based on a depth and a direction of the first echo signal, wherein the analog gain is automatically adjusted based on a peak amplitude of a second echo signal in a preceding ultrasound image.

2. The method of claim 1, wherein the second echo signal originates from the same depth and direction and covers a same spatial neighborhood as the first echo signal.

3. The method of claim 1, further comprising generating an ultrasound image based on the first echo signal and displaying the ultrasound image on a display.

4. The method of claim 1, wherein adjusting the analog gain based on the peak amplitude comprises calculating a difference, via a digital processor, between the peak amplitude and a reference amplitude, and subtracting a value proportional to the difference from an analog gain applied to the second echo signal.

5. The method of claim 1, wherein the analog gain is further adjusted based on limits of an analog-digital converter configured to digitize the first echo signal.

6. The method of claim 5, wherein adjusting the analog gain based on the limits comprises setting the analog gain to a maximum limit if the analog gain is above the maximum limit, and setting the analog gain to a minimum limit if the analog gain is below the minimum limit.

7. The method of claim 1, further comprising multiplying the second echo signal by a value inversely proportional to an instantaneous gain applied to the first echo signal.

8. A method for ultrasound imaging, comprising:
applying a first analog gain to a first echo signal based on the depth and direction of the first echo signal;
measuring a peak amplitude of the first echo signal;
adjusting a second analog gain applied to a second echo signal based on the peak amplitude;
generating a first ultrasound image based on the first echo signal and a second ultrasound image based on the second echo signal; and
displaying the first ultrasound image and the second ultrasound image in succession.

9. The method of claim 8, wherein measuring the peak amplitude is performed responsive to the first echo signal originating from a specified control point.

10. The method of claim 9, further comprising interpolating a third analog gain applied to a third echo signal based on the adjusted second analog gain.

11. The method of claim 8, wherein the first echo signal is converted to a first digital echo signal after applying the first analog gain and prior to measuring the peak amplitude.

12. The method of claim 8, wherein the second echo signal is converted to a second digital echo signal after applying the second analog gain and prior to generating the second ultrasound image.

13. The method of claim 8, wherein generating the first and second ultrasound images comprises applying digital beamforming techniques respectively to the first echo signal and the second echo signal.

14. The method of claim 8, wherein displaying the ultrasound images comprises transmitting the ultrasound images to a display device.

15. The method of claim 8, further comprising multiplying the first echo signal by a value inversely proportional to an instantaneous gain applied to the second echo signal prior to generating the first ultrasound image.

16. An ultrasound imaging system, comprising:
a transducer array including a plurality of array elements, the transducer array adapted to transmit a plurality of ultrasound waves and receive a plurality of echoes;
a display device configured to display an ultrasound image;
a gain controller comprising a memory, the memory configured with an analog gain matrix, the gain controller configured to apply an analog gain output by the memory to each of the plurality of echoes; and
a processor configured with computer-readable instructions in non-transitory memory that when executed cause the processor to update the analog gain matrix based on a peak amplitude of each of the plurality of echoes and generate the ultrasound image based on the plurality of echoes.

17. The system of claim 16, wherein the analog gain matrix comprises a table of analog time gain compensation values, wherein each of the analog time gain compensation values corresponds to a particular range and vector number.

18. The system of claim 17, wherein the gain controller further comprises a counter configured to provide a range of each of the plurality of echoes to the memory, and wherein the analog gain applied to each of the plurality of echoes is based on the range of each of the plurality of echoes.

19. The system of claim 16, wherein the gain controller further comprises a digital-analog converter configured to convert a digital gain value from the memory into the analog gain.

20. The system of claim 16, wherein the processor is further configured with computer-readable instructions in the non-transitory memory that when executed cause the processor to compute a first adjusted analog gain based on a specified echo and interpolate a second adjusted analog gain based on the first adjusted analog gain, and wherein updating the analog gain matrix comprises recording the first adjusted analog gain and the second adjusted analog gain to the memory.
